(19) United States
(12) Patent Application Publication
(10) Pub. No.: US 2022/0354577 A1
(43) Pub. Date: Nov. 10, 2022

(54) DEVICE, SYSTEM AND METHOD FOR REMOVAL OF HAIR

(71) Applicant: EPILADY 2000 LLC, Hazor Haglilit (IL)

(72) Inventor: Yehuda PORAN, Ramat Hagolan (IL)

(73) Assignee: EPILADY 2000 LLC, Hazor Haglilit (IL)

(21) Appl. No.: 17/774,141

(22) PCT Filed: Nov. 8, 2020
(86) PCT No.: PCT/IL2020/051158

§ 371 Date: May 3, 2022

(30) Foreign Application Priority Data
Nov. 8, 2019 (EP) 19208055.4

(51) Int. Cl. A61B 18/20

(52) U.S. Cl. CPC A61B 18/203; A61B 2018/00476

(57) ABSTRACT

A device, system and method for target localization is disclosed. An image sensing system generate and store a target data and communicate it to an adjustment engine. The adjustment engine further focuses radiations from a laser system to the target. The present invention further discloses a proximity sensing engine which is configured to enable control the radiation from the laser system. This enable for an efficient use of the radiation energy and reduces the potential damage to the area near the target.

### FIELD

The present invention is directed to a device, method, system and an apparatus for localizing and removing hair or tattoos.

### BACKGROUND

For hair removal, several methods and corresponding devices are known in the art, such as laser-based hair removal and intense pulsed light (IPL).

When skin is exposed to appropriate radiation, the radiation penetrates the skin. Depending on their absorption coefficient and the wavelength of the radiations most of the energy is absorbed by the chromophores which are consequently damaged. This damage is especially magnified when there is a significant difference between the skin's and the chromophore's absorption coefficient. The energy is absorbed by chromophores, which are either endogenous in the tissues, such as melanin, haemoglobin, etc or the exogenous compounds, such as different colours of tattoo ink.

By using radiation of a particular wavelength that is absorbed by the chromophores, the damage can be substantially limited to the hair follicles or tattoo ink. For providing radiation of a defined wavelength, laser-technology is known in the art.

The following documents discuss the use of laser light or laser devices for hair removal:

U.S. Pat. No. 6,030,378 A discloses a method of hair removal, used primarily for cosmetic purposes, comprising the transcutaneous use of laser light having a wavelength which targets the sebum found in the follicle and coating the hair, heating the sebum which transfer heat first to the hair and hair root and then to the papilla and papillary blood vessels via conduction, thus destroying the hair by photo thermolysis while avoiding significant damage to surrounding skin or tissue.

U.S. Pat. No. 6,666,856 B2 discloses a hair removal device which includes a cooling surface which is used to contact the skin prior to exposure to hair tissue-damaging laser light passing from a radiation source through a recessed window. The window is laterally offset from the cooling surface and is spaced apart from the cooling surface in a direction away from the patient's skin to create a gap between the window and the skin. The window preferably includes both an inner window and an outer, user-replaceable window. The laser-pulse duration is preferably selected according to the general diameter of the hair.

U.S. Pat. No. 7,029,469 B2 discloses a method and apparatus for removing hairs from living skin. The method and the device are involving the measurement with a colorimeter of the colour of the area of the skin where the hair is to be removed to obtain a representative colour value, employing the colour value to select an optimum range of laser energy necessary to inactivate hair follicles in the area, yet minimize any inflammatory reaction, and directing laser energy of optimum range at the skin area to depilate such area.

U.S. Pat. No. 7,108,690 B1 discloses a hair-removing device that includes a laser source, an adjustable laser beam manipulator for positioning a laser beam of the laser source in a target position on a skin to be treated, and an image sensor for detecting an image of the skin. The hair-removing device further comprises a control unit which determines a position and orientation on the skin of a hair to be removed, and which determines the target position of the laser beam as a function of said position and orientation of the hair. The control unit brings the laser beam manipulator in a state corresponding to the target position of the laser beam, and activates the laser source when the laser beam manipulator has reached said state. Thus, the hair-removing device is suitable for use by inexperienced users, and is particularly suitable for the consumer market. In a particular embodiment, the control unit determines the target position of the laser beam in a position on the skin under which a root of the hair is present, so that the root of the hair is destroyed and the hair-removing device is an epilating device by means of which the hair is removed for a relatively long time or even permanently. In another embodiment, the control unit determines the target position of the laser beam in a position on the hair where the hair comes out of the skin, so that the hair is burnt through near the skin surface and the hair-removing device is a shaving device by means of which a high skin smoothness is obtained.

Furthermore, the use of intense pulsed light is known in the art. Intense-pulsed-light epilation works as described above, however, as the pulsed light is no Laser light, it is typically not limited to a particular wavelength or to a narrow interval of wavelengths. Thus, the energy may be applied as selectively to the hair follicles and/or colours of tattoo ink. However, these devices can be smaller and even handheld. US 20140236136 A1 discloses a device and method of hair removal treatment. The device includes a combination of two light sources one an IPL and second an incandescent lamp type, halogen or others. The two light sources are applied simultaneously or sequentially to the treated area. The present invention relates to a method for hair removal treatment where multiple light sources are combined together and include at least one pulsed arc lamp, and are operated simultaneously or sequentially to treat a selected skin area. The incandescent light energy will raise the overall skin temperature while the arc lamp will selectively ablate the hair follicle with minimum collateral damage.

### SUMMARY

in light of the above, it is an object of the present invention to overcome or at least alleviate the shortcomings of the prior art. More particularly, it is an object of the present invention to provide a device, method, system and an apparatus for localizing a target based on a type of a target area, such as skin colour, colour of tattoo ink, etc. It is a further object of the present invention to create a secure environment for damaging the target using the laser technology.

It Is also an object of the present invention to provide a system that is configured to identify a target and further adjust a lens and/or a laser system to focus a beam on the target with increased efficiency and decreasing the potential damage to the area nearby.

These objects are fulfilled by the device, apparatus, method and system of the present invention.

In a first embodiment a device for target localization is disclosed. The device comprises a laser system to emit radiation beams. The radiation beams may be parallel to each other. The plurality of radiation beam is then modulated by an adjustment engine. The adjustment engine comprises a lens and is configured to move the lens in respect to the laser system such that the target position lies on an optical axis of the lens. The radiation beams are then focused by the lens at a focal region on the optical axis. The device further comprises an image sensing system which is configured to generate target data.

In some embodiments the device comprises a processing component. The processing component may be an electric circuitry which can be configured to carry out instructions by performing arithmetic, logic, controlling and input/output operations. The processing component may also comprise a microprocessor and, in some embodiments, a single integrated circuit, PCB etc. The processing component may also comprise a communication component which can be configured to enable a bilateral data exchange with the image sensing system and/or the adjustment engine. In a further embodiment the processing component may be configured with a signal processing component.

In some embodiments the communication component may further be configured to enable a bilateral data exchange with a user terminal. The user terminal may be a personal computer, laptop, mobile app, etc. In a further embodiment the processing component may also comprise a memory component. The memory component may be storing data temporarily or permanently. In some embodiments the memory component may be volatile and/or non-volatile.

In some embodiments the laser system may comprise a laser source and/or a plurality of laser sources. Each laser source may comprise a laser diode or a plurality of laser diodes, injection laser diode or diode laser. The laser diode may comprise different semiconductor material determining the wavelength of the emitted beam. Each laser sources may be configured with a different wavelength. In some embodiments the processing component may control the laser system and based on the target data the wavelength can be chosen. For example, a laser of wavelength 755 nm can be used if the target area is fair to mid-toned skin tissues with target being medium to coarse dark hair. The diode laser in the range of 800 nm to 810 nm may be used for fair to mid-toned skin with dark fine hair. In some embodiments the laser system may be configured to combine a plurality of laser sources with different wavelengths in a single emission. The laser system may further be configured to program the fluence and/or pulse duration of an emission to increase the safety and effectiveness. In a further embodiment the laser system comprises at least four laser sources. Each laser sources comprising same or similar wavelengths. In another embodiment each laser sources may comprise a different wavelength. The laser system may further comprise at least one pair of laser sources, wherein the pair of laser sources may comprise the lasers with same wavelengths. The laser sources may be configured to be mounted on a laser system plane.

In a further embodiment the processing component may control the fluence and wavelength of the laser emission from the laser system based on the target data. The wavelength of each laser source may be in the range of 590 to 710 nm, preferably 610 to 680 nm, such as 630 to 650 nm, more preferably 640 nm. In another embodiment the wavelength of each laser sources may be in the range of 710 nm to 880 nm, preferably 740 to 850 nm, such as 790 to 810 nm, more preferably 808 nm. In a further embodiment the wavelength of each laser sources may be in the range of 880 to 1200 nm, preferably 910 to 1105 nm, such as 930 nm to 990 nm, more preferably 980 nm.

In some embodiments the at least two laser sources from a laser pair may be configured to be mounted on the laser plane. The two laser sources may be mounted opposite to each other in the laser system plane, in such embodiments the two different pairs of laser sources emitting different wavelengths may create a focal area once focused at an optical axis by a lens. The lens may be configured to be placed between the laser system and the target area. In some embodiments the two laser sources may have the same wavelength resulting in a focal point on the optical axis. The focal area may comprise a plurality of focal points along the length of the optical axis of the lens. In some further embodiments a plurality of laser source pairs may be configured to be emitting radiations at the lens peripheral area, the laser sources may be displaced by, such as 180°. In such embodiments this plurality of laser source pairs may be configured with different wavelengths configured to create a long focal area resulting in increased efficacy. In some further embodiments the lens may comprise a lens system configured with a plurality of lenses.

In some embodiments the processing laser source may be configured to deliver a fluence in the range of 10 to 40 J/cm, such as 20 to 30 J/cm², preferably 22 to 25 J/cm², more preferably 24 J/cm. In some embodiments the processing component may be configured to change the fluence of the laser source based on the target. For example, if the target is skin tissues and the user terminal has inputted a user data for the skin being sensitive then the processing component may emit lower fluence. In a further embodiment the fluence may be reduced by to 65% and the emission may be used as a targeting system or for illumination of the target. In some embodiments the processing component may be configured to adjust the fluence based on a target size.

In some embodiments the adjustment engine may be configured to change a relative position of a lens with the laser system. The adjustment engine may comprise two linear devices, preferably perpendicular to each other. The two linear devices may be configured to be actuated simultaneously. The two linear devices may be configured to hold the lens and move the lens preferably in two perpendicular directions. This movement of the lens by the adjustment engine is configured to move an optical axis of the lens and hence the focal point. In further embodiments, the processing component may be configured to control the adjustment engine based on the target data. The target data may comprise coordinate axes associated with the target. The adjusting engine may further comprise displacing the optical axis when the radiations are received by the image sensing system so as to not block the received radiations.

In some embodiments the device may comprise an aperture. The aperture may be configured to let radiation from the laser system exit the device, preferably when the device is close to the target area, such as skin. The aperture may be comprising a plurality of shapes. The processing component may be configured to generate an aperture shape based on the target data. For example, the processing component may be configured to send the user terminal the information about the shape and size of the aperture to use based on the target.

In some further embodiments the aperture may comprise a cover. The cover may be transparent to the radiations from the laser system. In some embodiments the cover may be 80 to 70% transparent. In some further embodiments the cover may be configured to change the fluency of the radiation from the laser system. The cover may be semi or fully transparent to the radiations received by the image sensing system.

In some embodiments the image sensing system comprises a camera. The camera may be configured to sense at least one target data and transmit it to the processing component. The image sensing system may further be sensing a plurality of image data that are generated at different positions of the optical axis. The processing component may be configured to determine at least one position and/or at least one type of the target based on the received target data. In a further embodiment the device may comprise a targeting system. The targeting system may be configured to deliver a plurality of EM radiations in different parts of the wavelengths. The targeting system may be configured on a periphery of the aperture of the device so as to not hinder the radiations from the laser system. The EM radiations from the targeting system may be reflected by a background object, such as flesh resulting in backlighting the target area. The targeting system may be emitting the EM radiations such that the angle of incidence with the target area is in the in the range of 75° to 105°, preferably 90° to the target area. Further, the angle of reflection with respect to the background object may be in the range of 35° to 95°. The image sensing system can be configured to only receive the reflected EM radiations. The image sensing further senses at least one backlighted target area and transmit the sensed target area it to the processing component.

In a further embodiment the processing component may be further configured with machine learning techniques, for example, image recognition techniques. In such embodiments the processing component may receive the backlighted target area and may classify the target in classes, for example, on the basis of the target colour. In some further embodiments wherein the targeting system may be configured to emit acoustic radiations and the processing component may be configured to classify the at least one target based on the reflected radiations.

In a second embodiment an apparatus for sensing target proximity is disclosed. The apparatus comprises a proximity sensing engine, a processing component and an output engine. The output engine may be configured to enable a laser system only when a safety value is transmitted to the output engine by the processing component. The processing component may be configured to calculate a safety value based on the proximity data transmitted by the proximity sensing engine. The proximity sensing engine may be configured with at least one or a plurality of capacitive sensing device(s) and/or photoelectric sensing device(s) and/or electromagnetic induction sensing device(s). The proximity sensing engine may further be configured with at least one or a plurality of accelerometer(s) and/or gyroscope(s), compass(s). The proximity sensing engine may be configured at the perimeter of the aperture. In some embodiments the proximity sensing engine may be configured on an upper body the device. The proximity sensing engine may further generate the proximity data based on gravity, magnetic orientation and/or distance from a target area. The proximity sensing engine may be configured to detect the motion of the apparatus and/or the body in respect to the body and/or apparatus. In some further embodiments the processing component may be configured to only generate the safety value when the proximity data is within a pre-determined range. This Is advantageous when the user is not placing the apparatus near the skin. The apparatus will only be activated once the target area is in the safe distance.

In a further embodiment the apparatus may comprise an energy storage component, such as a battery. The energy storage component may be a lithium-ion battery or the alike. In some embodiments the apparatus may be configured with a charging connector. In some further embodiments the output engine may be configured to send an output signal to a user terminal. The output signal may be an acoustic output signal.

In a third embodiment a system can be configured to localise a target is disclosed. The system comprises the device according to the preceding description and the apparatus according to the preceding description. The system is configured to enable the laser system once the processing component transmits the safety value.

In a fourth embodiment a method for localising a target is disclosed, especially by using a laser system according to any of the relevant preceding claims.

The invention is further described with the following numbered embodiments.

Below, system embodiments will be discussed. These embodiments are abbreviated by the letter “D” followed by a number. Whenever reference is herein made to “device embodiments”, these embodiments are meant.
D1. A device for target localization, comprising:
   (a) a laser system;
   (b) an adjustment engine configured to displace an optical axis of a lens system; and
   (c) an image sensing system configured to generate a target data.
D2. The device according to the preceding embodiment wherein the device further comprises a processing component configured to transmit the target data to the adjustment engine.
D3. The device according to any of the preceding embodiments wherein the device is configured to send data to a user terminal.
D4. The device according to any of the preceding embodiments wherein the device is further configured to receive data from the user terminal.
D5. The device according to any of the two preceding embodiments, wherein the device comprises a communication component that is configured to send and/or receive data to/from the user terminal.
D6. The device according to any of the preceding embodiments with the features of D2 wherein the processing system comprises at least one memory component.
D7. The device according to any of the preceding embodiments wherein the laser system comprises at least one laser source.
D8. The device according to any of the preceding embodiments wherein the laser system comprises a plurality of laser sources.
D9. The device according to any of the preceding embodiments wherein the laser system comprises four laser sources.
D10. The device according to any of the preceding device embodiments with the features of D7, wherein each laser source comprises a laser diode component.
D11. The device according to any of the preceding device embodiments wherein each laser source comprises a plurality of a laser diode component.
D12. The device according to any of the preceding embodiments wherein a wavelength of each laser source is in the range of 590 to 710 nm, preferably 610 to 680 nm, such as 630 to 650 nm, more preferably 640 nm.
D13. The device according to any of the preceding embodiments wherein the wavelength of each laser source is in the range of 710 nm to 880 nm, preferably 740 to 850 nm, such as 790 to 810 nm, more preferably 808 nm.
D14. The device according to any of the preceding embodiments wherein the wavelength of each laser source is in the range of 880 to 1200 nm, preferably 910 to 1105 nm, such as 930 nm to 990 nm, more preferably 980 nm.
D15. The device according to any of the preceding three embodiments wherein at least two laser sources have different wavelengths.
D16. The device according to any of the preceding embodiments wherein the wavelength of at least two laser sources are the same or similar.
D17 The device according to any of the preceding embodiments wherein the laser system is configured to focus the laser sources at a focal point.
D18. The device according to the preceding embodiment wherein the focal point is configured to be on an optical axis of the lens system.
D19. The device according to any of the preceding embodiments wherein the lens system comprises at least one lens.
D20. The device according to any of the preceding embodiments wherein the lens system comprises at least one plurality of lenses.
D21. The device according to the preceding embodiment wherein the plurality of lenses is configured with a common optical axis.
D22. The device according to any of the preceding embodiments wherein the at least two lenses comprises different optical axes, preferably the optical axes parallel to each other.
D23. The device according to any of the preceding embodiments wherein the lens system is further configured to converge the radiation from the laser system to a focal area.
D24. The device according to the preceding embodiment wherein the focal area comprises a plurality of the focal points.
D25. The device according to the preceding embodiment wherein the focal points are configured to be along the optical axis of the lens system.
D26. The device according to any of the preceding embodiments wherein the focal area comprises at least two laser sources with different wavelengths.
D27. The device according to any of the preceding embodiments wherein the at least two laser sources are configured to be installed on at least one laser system plane.
D28. The device according to the preceding embodiment wherein the laser system plane is configured to be perpendicular to the optical axis of the lens system.
D29. The device according to any of the preceding embodiments wherein at least two laser sources are configured to emit radiation parallel to the optical axis of the lens system.
D30. The device according to any of the preceding embodiments wherein the at least two laser sources are configured to be installed on the at least one laser system plane at a distance in a range of 0.8 cm to 2.10 cm, preferably 1 cm to 1.5 cm, such as 1.65 cm opposite to each other.
D31. The device according to any of the preceding embodiments wherein each laser source is configured to deliver a fluence in the range of 10 to 40 J/cm², such as 20 to 30 J/cm², preferably 22 to 25 J/cm², more preferably 24 J/cm².
D32. The device according to any of the preceding embodiments wherein the fluence of each laser source is configured to be decreased by 45 to 65%.
D33. The device according to the preceding embodiment wherein the laser system is configured to be used as a targeting system.
D34. The device according to any of the preceding embodiments wherein the adjustment engine is configured to change a relative position of at least a portion of the laser system to the lens system and/or to the lens system's optical axis.
D35. The device according to any of the preceding embodiments wherein the adjustment engine comprises two linear drives.
D36. The device according to the preceding embodiment wherein the two linear drives can be actuated simultaneously.
D37. The device according to any of the preceding embodiments wherein the processing system is configured to control the adjustment engine or at least a portion thereof.
D38. The device according to any of the preceding embodiments wherein the device comprises an aperture that is configured to let radiation from the laser system exit the device, preferably via an opening.
D39. The device according to any of the preceding embodiments wherein the opening is configured to have an opening width in a range of 1 cm to 2 cm, such as 1.5 cm.
D40. The device according to any of the preceding embodiments wherein an aperture plane is configured to be preferably perpendicular the optical axis of the lens system.
D41. The device according to any of the preceding embodiments wherein the aperture plane and the lens system plane are configured to have a distance in a range of 2 cm to 6 cm, preferably 3 cm to 5 cm, such as 4.5 cm.
D42. The device according to the preceding embodiment wherein the aperture comprises a cover.
D43. The device according to the preceding embodiment wherein the cover is transparent to the radiation from the laser system.
D44. The device according to any of the two preceding embodiments wherein the cover is transparent to radiation that is received by the image sensing system.
D45. The device according to any of the preceding embodiments wherein the image sensing system comprises a camera.
D46. The device according to any of the preceding embodiments wherein the image sensing system is configured to save target data using a plurality of pictures that are generated at different positions of the optical axis.
D47. The device according to any of the preceding embodiments with the features of D2 wherein the processing system is configured to exchange data with the image sensing system.
D48. The device according to the preceding embodiment wherein the processing system is configured to determine a position of a target from the target data.
D49. The device according to the preceding embodiment wherein the processing system is further configured to control the adjustment engine and the laser system according to the position of the target.
D50. The device according to any of the preceding embodiments with the features of D2, D4 and D5, wherein the image sensing system is further configured to exchange data with the user terminal.
D51. The device according to the preceding embodiment wherein the device is configured to pull the target data from the user terminal and wherein the processing system is furthermore configured to control the adjustment engine and the laser system based on the pulled target data.
D52. The device according to any of the preceding embodiments wherein the memory component is configured to store the target data.
D53. The device according to the preceding embodiment wherein the target data comprise positions of hair follicles relative to the aperture.
D54. The device according to any of the preceding embodiments wherein the target data comprise positions of ink pigments relative to the aperture.
D55. The device according to any of the preceding embodiments wherein the target data comprises positions of the skin cells with pigmentation value higher than a threshold value.
D56. The device according to the any of the preceding embodiments wherein the target data comprise instruction data for controlling at least one of the at least the adjustment engine and at least the laser system.
D57. The device according to any of the preceding embodiments wherein the device further comprises the targeting system.
D58. The device according to the preceding embodiment wherein the targeting system comprises a plurality of sources configured to provide electromagnetic (EM) radiations in different parts of the wavelength spectrum.
D59. The device according to any of the preceding embodiments wherein each source comprises a LED component.
D60. The device according to the preceding embodiment wherein the source is configured to send electromagnetic radiations which is further selectively or fully reflected by a background object.
D61. The device according to the preceding embodiment wherein the angle of reflection is in the range of 35° to 95°.
D62. The device according to the preceding embodiment wherein the source is configured to send EM radiations at an angle of incidence in the range of 75° to 105°, preferably 90°.
D63. The device according to any of the preceding embodiments wherein the targeting system is configured to be at an outer periphery of the aperture and/or the opening.
D64. The device according to any of the preceding embodiments wherein the aperture is configured to enclose the target area.
D65. The device according to any of the preceding embodiments wherein the reflected EM radiation is configured to illuminate a background area of a target area.
D66. The device according to any of the preceding embodiments wherein the image sensing system is configured to capture the illuminated target area.
D67. The device according to any of the preceding embodiments wherein the image sensing system is further configured to generate the target data using the captured illuminated target area.
D68. The device according to the preceding embodiment wherein the image sensing system is configured to calculate at least one absorption value of the target.
D69. The device according to any of the preceding embodiments wherein the processing component is configured with machine learning techniques, such as deep learning for image recognition.
D70. The device according to the preceding embodiment wherein the processing component is configured to classify the target data, preferably using image recognition.
D71. The device according to any of the preceding embodiments wherein the targeting system is configured to emit at least one acoustic radiation.
D72. The device according to the preceding embodiment wherein the image sensing system is configured with machine learning techniques to recognize the target data based on reflected acoustic radiations.
D73. The device according to any of the preceding embodiments wherein the device is configured to estimate a colour of the target area by the image sensing system, preferably using the absorption value.
D74. The device according to any of the preceding embodiments wherein the processing system is configured to control at least one of the at least the adjustment engine and at least the laser system based on the estimated colour(s).

Below, apparatus embodiments will be discussed. These embodiments are abbreviated by the letter W followed by a number. Whenever reference is herein made to “apparatus embodiments”, these embodiments are meant.
A1. An apparatus for sensing target proximity comprising:
   (a) a proximity sensing engine;
   (b) a processing component; and
   (c) an output engine.
A2. The apparatus according to any of the preceding embodiments wherein the output engine is configured to enable a laser system only when a safety value is transmitted to the output engine by the processing component.
A3. The apparatus according to any of the preceding embodiments wherein the proximity sensing engine is configured to send the proximity data to the processing component.
A4. The apparatus according to any of the preceding embodiments wherein the processing component is configured to automatically generate a safety value on the basis of the proximity data.
A5. The apparatus according to any of the preceding embodiments wherein the proximity sensing engine comprises at least one or a plurality of capacitive sensing device(s) and/or photoelectric sensing device(s) and/or electromagnetic induction sensing device(s).
A6. The apparatus according to any of the preceding embodiments wherein the proximity sensing engine comprises at least one or a plurality of accelerometer(s).
A7. The apparatus according to any of the preceding embodiments wherein the proximity sensing engine comprises at least one or a plurality of gyroscope(s).
A8. The apparatus according to any of the preceding embodiments wherein the proximity sensing engine comprises at least one or a plurality of compass(s).
A9. The apparatus according to any of the preceding embodiments wherein the proximity sensing engine is configured to generate the proximity data based on gravity, magnetic orientation and/or distance from a target area.
A10. The apparatus according to the preceding embodiment wherein the proximity sensing engine is configured to detect a proximity of a human body to an aperture of the apparatus.
A11. The apparatus according to the preceding embodiment wherein the proximity sensing engine is further configured to detect the motion of the body in respect to the device.
A12. The apparatus according to any of the preceding embodiments wherein the processing component generates the safety value only when the proximity data is within a pre-determined range.
A13. The apparatus according to any of the preceding embodiments wherein the apparatus comprises an energy storage component, such as a battery.
A14. The apparatus according to the preceding embodiment wherein the energy storage component is a lithium-ion battery.
A15. The apparatus according to any of the preceding embodiments wherein the apparatus comprises a charging connector.
A16. The apparatus according to any of the preceding embodiments wherein the output engine is configured to send an output signal to a user terminal.
A17. The apparatus according to the preceding device embodiment wherein the output engine is configured to send an acoustic output signal to the user terminal.
A18. The apparatus according to any of the preceding embodiments wherein the output engine is configured to send the output signal to the user at least when the laser system and/or the adjustment engine is operated.
   Below, system embodiments will be discussed. These embodiments are abbreviated by the letter “S” followed by a number. Whenever reference is herein made to “system embodiments”, these embodiments are meant.
S1. A system configured to localise a target with safety, comprises:
   (a) a device for target localization according to any of the preceding device embodiments;
   (b) an apparatus for sensing target proximity according to any of the preceding apparatus embodiments;
   wherein, the system is configured to enable the device based on a safety value generated by the apparatus.
S2. The system according to the preceding embodiment wherein the device comprises a plurality of laser sources.
S3. The system according to the preceding embodiment wherein the at least two laser sources have different wavelengths.
S4. The system according to any of the preceding embodiments wherein the system is further configured to deliver a plurality of laser sources at a focal point on an optical axis of a lens system.
S5. The system according to any of the preceding embodiments wherein the system is further configured to deliver a plurality of laser sources at a focal area along the optical axis of the lens system.
S6. The system according to any of the preceding embodiments wherein the system is further configured to change a position of the lens system relative to the laser source.
S7. The system according to any of the preceding embodiments wherein the system comprises a processing component.
S8. The system according to any of the preceding embodiments wherein the processing component is configured to generate target data.
S9. The system according to any of the preceding embodiments wherein the system further comprises an image sensing system.
S10. The system according to the preceding embodiment wherein the image sensing system is configured to receive at least one target area data.
S11. The system according to any of the preceding embodiment wherein the processing component is configured to pull target area data from the image sensing system.
S12. The system according to any of the preceding embodiments wherein the processing component is configured to control the laser sources.
S13. The system according to any of the preceding embodiments wherein the processing component change the position of the lens system based on the target data.
S14. The system according to the preceding embodiment wherein the apparatus comprises at least one or a plurality of capacitive sensing device(s).
S15. The system according to the preceding embodiment wherein the system comprises at least one of a at least one accelerometer and at least one gyroscope and at least one compass configured to generate the safety value.
S16. The system according to any of the preceding embodiments wherein the safety value comprises an alignment data of an aperture with the target area.
S17. The system according to any of the preceding embodiments wherein the system is further configured to enable the laser sources only when the alignment data is within a pre-determined range.
   Below, method embodiments will be discussed. These embodiments are abbreviated by the letter “M” followed by a number. Whenever reference is herein made to “method embodiments”, these embodiments are meant.
M1. A method for localizing a target comprising:
   (a) automatically identifying a target type and a target position;
   (b) generating a radiation based on the target type;
   (c) focusing the radiation based on the target position;
   (d) enabling the radiation only when a safety value is received.
M2. The method according to the preceding embodiment wherein the method comprises the step of generating radiations via a laser system configured with a laser source.
M3. The method according to the preceding embodiment wherein the method comprises the step of generating radiations via the laser system configured with a plurality of laser sources.
M4. The method according to any of the preceding embodiments wherein the method comprises the step of facilitating the at least two laser sources with a different wavelength.
M5. The method according to any of the preceding embodiments wherein the method comprises the step of facilitating the at least two laser sources with same or similar wavelengths.
M6. The method according to any of the preceding embodiments wherein the method further comprises adjusting a position of a lens system relative to the position of the laser source.
M7. The method according to any of the preceding embodiments wherein the method comprises the step of converging the radiations from the laser source to a focal point along an optical axis of the lens system.
M8. The method according to any of the preceding embodiments wherein the method comprises the further step of converging the radiations from the plurality of laser source to a focal point along the optical axis of the lens system.
M9. The method according to any of the preceding embodiments wherein the method comprises the step of converging the radiations from the plurality of laser sources with different wavelengths to a focal area along the optical axis of the lens system.
M10. The method according to any of the preceding embodiments wherein the wavelength of each laser source is in the range of 590 to 710 nm, preferably 610 to 680 nm, such as 630 to 650 nm, more preferably 640 nm.
M11. The method according to any of the preceding embodiments wherein the wavelength of each laser source is in the range of 710 nm to 880 nm, preferably 740 to 850 nm, such as 790 to 810 nm, more preferably 808 nm.
M12. The method according to any of the preceding embodiments wherein the wavelength of each laser source is in the range of 880 to 1200 nm, preferably 910 to 1105 nm, such as 930 nm to 990 nm, more preferably 980 nm.
M13. The method according to any of the preceding embodiments wherein the fluence of each laser source is in the range of 10 to 40 J/cm², such as 20 to 30 J/cm², preferably 22 to 25 J/cm², more preferably 24 J/cm².
M14. The method according to any of the preceding embodiments wherein the method comprises automatically generating target data using a processing component.
M15. The method according to the preceding embodiments wherein the method comprises the further step of enabling a bilateral data exchange between the processing component and an image sensing system.
M16. The method according to the preceding embodiment wherein the image sensing system comprises a camera.
M17. The method according to any of the preceding embodiment wherein the method comprises the step of delivering electromagnetic (EM) radiation via a targeting system.
M18. The method according to the preceding embodiment wherein the targeting system comprises a plurality of sources configured to deliver EM radiations in different parts of a wavelength spectrum.
M19. The method according to any of the preceding embodiments wherein the method comprises the step of delivering the EM radiations at an angle of incidence in the range of 75° to 105°, preferably 90°.
M20. The method according to any of the preceding embodiments wherein the method further comprises the step of reflecting the EM radiations from a background at an angle of reflection in the range of 35° to 85°.
M21. The method according to any of the preceding embodiments wherein the method comprises the step of backlighting a target area via the reflected EM radiations from the background.
M22. The method according to the preceding embodiment wherein the method further comprises recording the backlighted target area, preferably using the image sensing system.
M23. The method according to the preceding embodiment wherein the method comprises the step of adjusting the position of the lens system prior to the recording.
M24. The method according to any of the preceding embodiments wherein the method comprises the step of automatically forwarding the recorded target area to the processing component.
M25. The method according to any of the preceding embodiments wherein the method further comprises processing the recorded target area to generate the target data.
M26. The method according to the preceding embodiment wherein the step of processing comprises applying signal processing techniques.
M27. The method according to any of the preceding embodiments wherein the step of processing comprises applying machine learning techniques.
M28. The method according to any of the preceding embodiments wherein the method further comprises the step of adjusting the lens system based on the target data.
M29. The method according to any of the preceding embodiments wherein the target data comprises target position and target type.
M30. The method according to any of the preceding embodiments wherein the method further comprises the step of communicating the target data from the processing component to the laser system.
M31. The method according to any of the preceding embodiments wherein the method comprises the step of facilitating the processing component with a proximity sensing engine.
M32. The method according to the preceding embodiments wherein the method comprises the step of processing component only sending the target data to the laser system once the proximity sensing engine has generated a safety value.
M33. The method according to any of the preceding embodiments wherein the method comprises automatically calculating safety value based on the orientation of the proximity sensing engine.
   Below, use embodiments will be discussed. These embodiments are abbreviated by the letter “U” followed by a number. Whenever reference is herein made to “use embodiments”, these embodiments are meant.
U1. Use of the system according to any of the preceding system embodiments for carrying out the method according to any of the preceding method embodiments.

### BRIEF DESCRIPTION OF FIGURES

Reference numbers used in figures:
1. Laser device
2. Handel
3. Operating panel
4. Power switch
5. On off induction LED
6. Battery indicator
10. Laser opening
11. Aperture
12. Targeting system
20. Operating panel assembly.
21. Upper body
22. Lower body
23. Cover
29. Battery
30. Mechanism assembly
32. X direction motor
32A. X direction spindle drive
32B. X direction spindle nut
33. Y direction bar
34 Y direction motor
34A. Y direction spindle drive
34B. Y direction spindle nut
35. Adjustment engine
36. X direction bar
37S. Y direction frame slot
38. lens frame holder
39. Lens frame
44. Camera CCD
45. Processing component
50. Laser system
51. lens
52. laser beam
56 Camera lens
60 Opening assembly
61 Opening body
62 Lighting and sensor PCB
63 Light diode WL 1
64 Light diode WL 2
65 Light diode WL 3
66 Proximity sensing engine
70 Skin
71 Target area
72 Target
74 Lighting rays
78 Skin and hair pitcher

The present invention will now be described with reference to the accompanying drawings, which illustrate embodiments of the invention. These embodiments should only exemplify, but not limit, the present invention.

FIG. 1 depicts an embodiment of a laser device;

FIG. 2 is a blown-up view of a laser device;

FIG. 3 is a blown-up view of a mechanism assembly;

FIG. 4 is a perspective view of a portion of a laser system;

FIG. 5 shows a perspective view of an adjustment engine;

FIG. 6 and FIG. 7 depict embodiments of a laser system;

FIG. 8 is a blown-up view showing a targeting system;

FIG. 9 depicts an embodiment of the image sensing system;

FIG. 10 and FIG. 11 shows another embodiment of the laser device.

### DETAILED DESCRIPTION OF FIGURES

It is noted that not all the drawings carry all the reference signs. Instead, in some of the drawings, some of the reference signs have been omitted for sake of brevity and simplicity of illustration. Embodiments of the present invention will now be described with reference to the accompanying drawings.

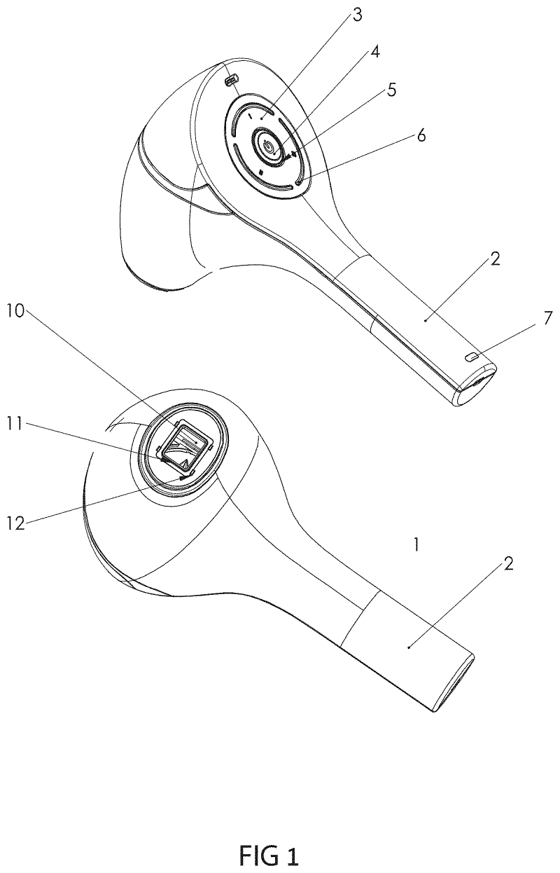

FIG. 1 is a schematic representation of the target localization device 1, a handle 2, an operating panel 3 which can be configured with a power switch 4. The device can further comprise a and/or a plurality of battery indicators 6. The battery indicators 6 can be configured to display a visual indication of the battery's state of charge (SoC) or depth of discharge (DoD). The battery indicator 6 may be an LED battery level indicator, or an electronic display taking the form of a bar graph. The device may further comprise an aperture 11 and a targeting system 12 preferably at the perimeter of the aperture 11. The battery indicators 6 may also be indicating the power of the lasers. The device may further comprise a laser opening 10 which may be configured to allow radiations pass.

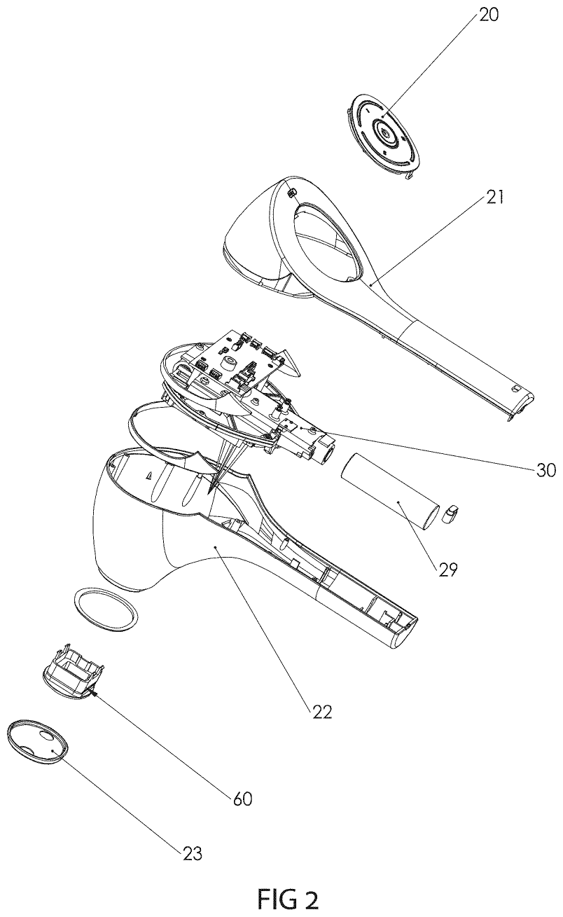

FIG. 2 shows a blown-up view of the laser device according to one embodiment of the present invention. The upper body 21 may comprise an operating panel assembly 20. The upper body 21 and the lower body 22 may be configured to be detachable. The mechanism assembly 30 may comprise the image sensing system, the laser system and the processing component. The battery 29 can be configured to fit in the handle 2. The battery may be a lithium-ion battery. The device may further comprise an opening assembly 60, the opening assembly may be configured to be in different shapes depending on a shape of a target area.

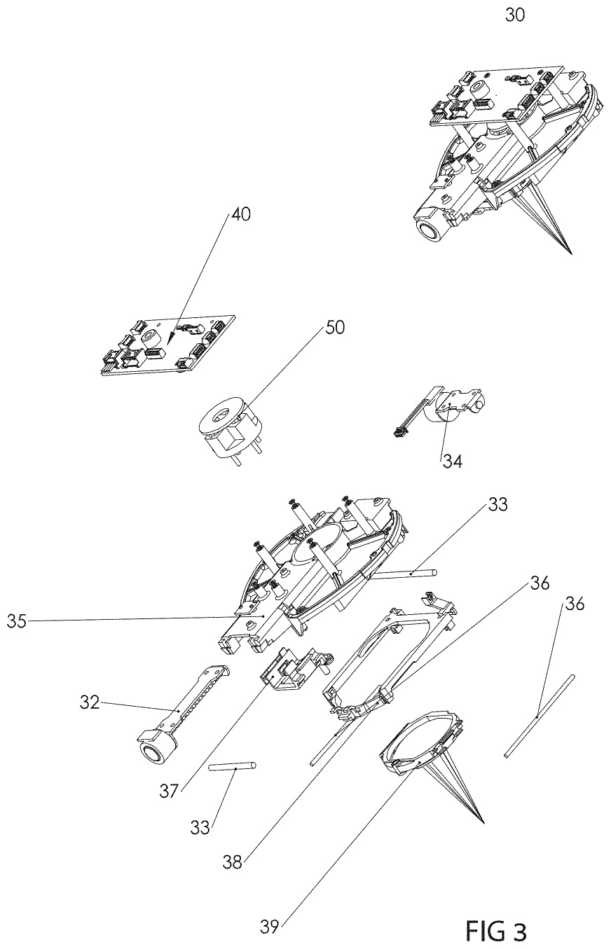

FIG. 3 shows a blown-up view of the mechanism assembly 30. The mechanism assembly 30 may comprise a processing component 45, a laser system 50 and, an adjustment engine 35.

The adjustment engine may comprise a X direction motor 32, a Y direction motor 34. The motors 32,33 may be configured to move a lens frame 39 along a x direction bar 36 and a y direction bar 33. Further, the device may comprise a x direction frame 37 and a lens frame holder 38.

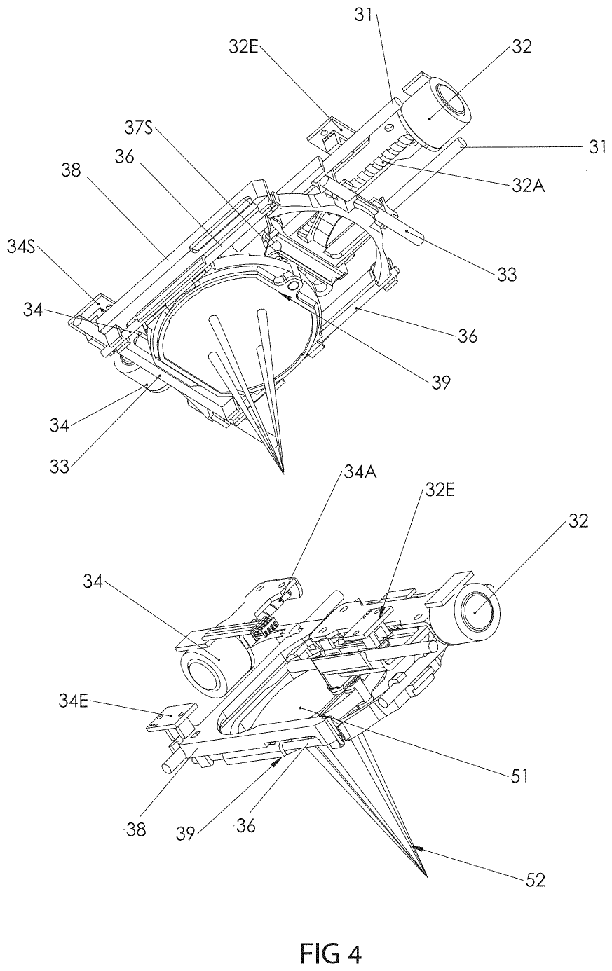

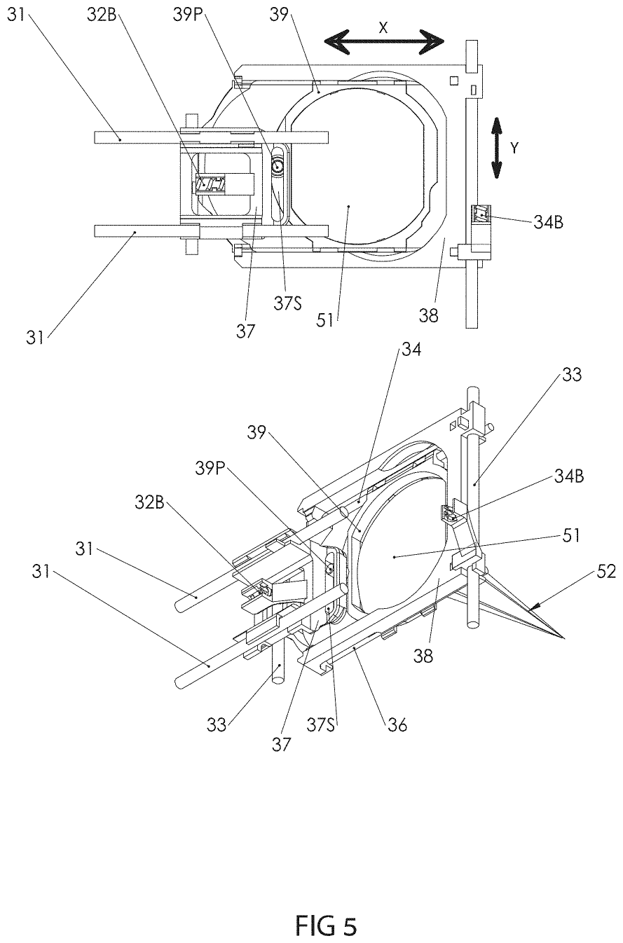

FIG. 4 and FIG. 5 shows a perspective view of a portion of the mechanism assembly 30 according to any embodiment of the present invention. The lens 51 may be enclosed in a lens frame 39. The lens frame 39 may further be mounted on two X direction bars 36. The lens frame 39 may also be configured to move between the two Y direction bars 33. The Y direction motor 34 may be driven by a Y direction spindle drive 34A. The Y direction spindle 34A drive may further be facilitated by the Y direction spindle nut 34B. The X direction motor 32 may be driven by a X direction spindle drive 32A which may be facilitated by a X direction spindle nut 32B. The Y direction motor 34 may further be configured to move on the Y direction frame bar 33 in the direction of the Y axis. The X direction motor 32 may further be configured to move on the X direction frame bar 31 in the direction of the X axis. The X direction frame 37 may comprise a X direction frame drive slot 37S along which a Y direction drive pin 39P may move. Hence, the lens frame 39 may move on both the X and the Y axis. Therefore, the lens 51 can move on two axes and cover the entire space of the laser opening 10.

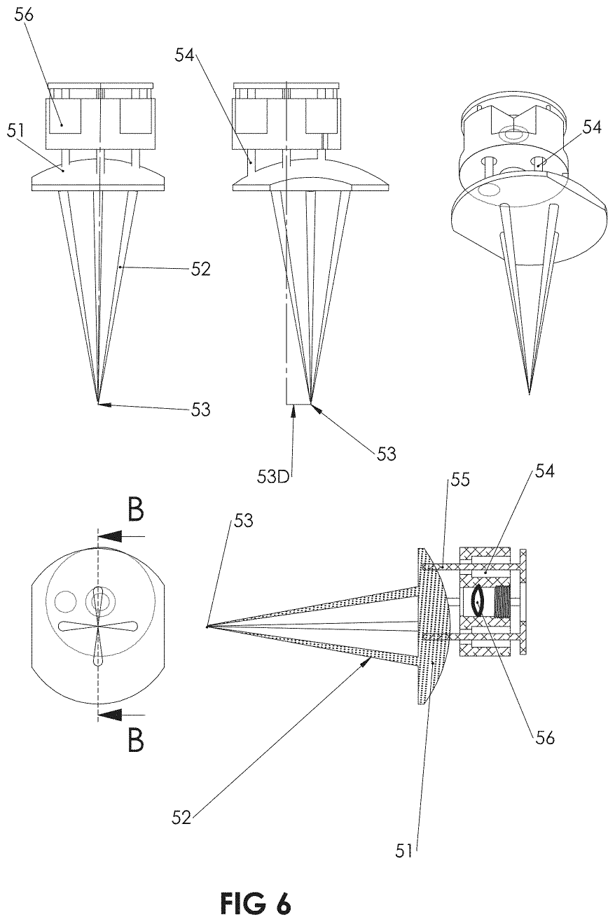

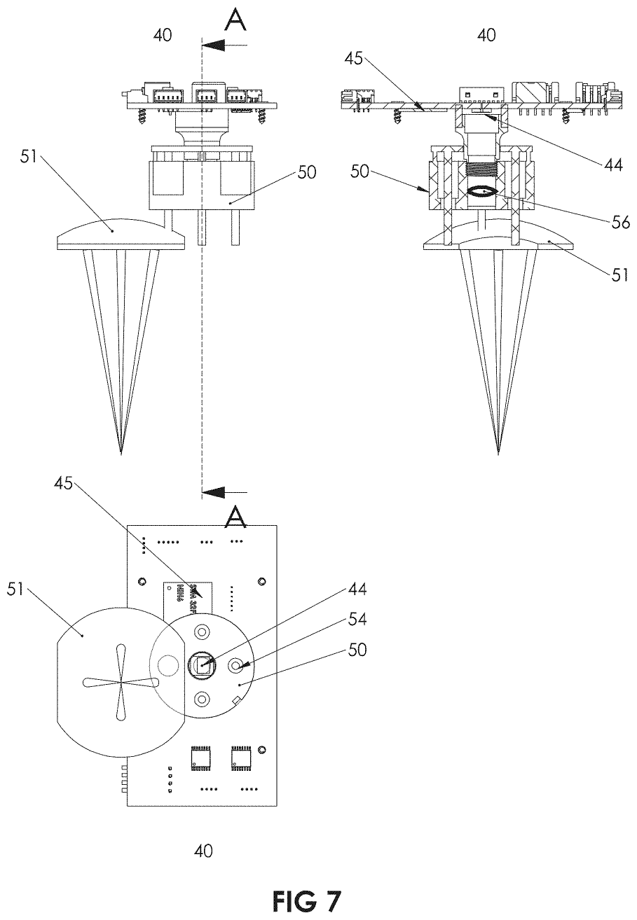

FIG. 6 and FIG. 7 depicts an embodiment of a laser system 50. The device may further comprise a lens 51 configured to focus 53 at least one or a plurality of radiation beams 52 from a laser diode 54. The radiation beams might be parallel 55 to each other The image sensing system may comprise the processing component 45. The processing component 45 may be a PCB assembly. The image sensing system may comprise a camera lens 56. The camera lens may be configured to take an image data from a target area. The target area data may then be pulled by processing component 45. The camera CCD 44 may be configured to enable at least one digital signal processing.

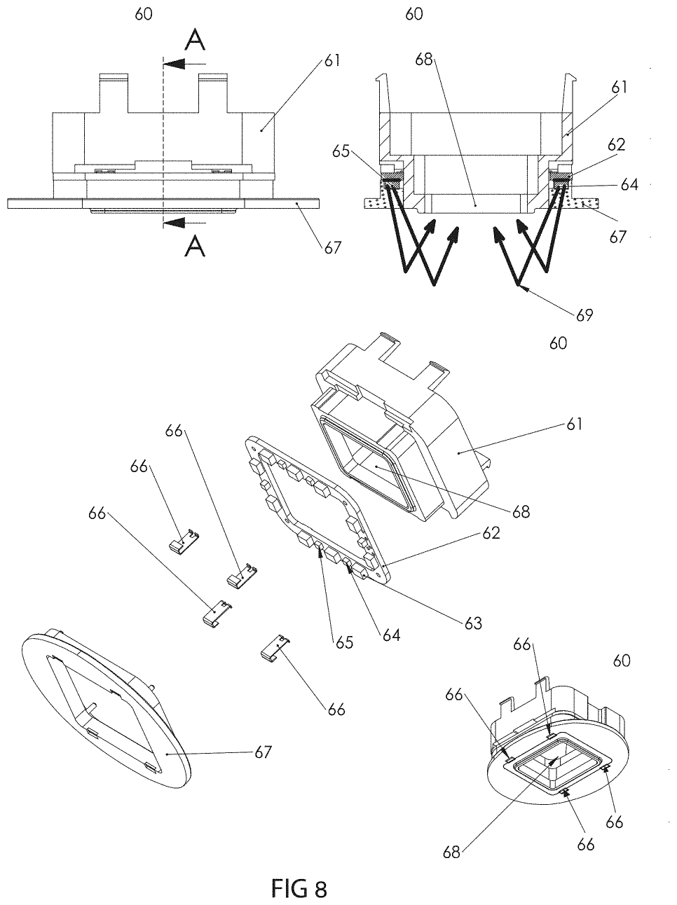

In FIG. 8 the opening assembly 60 is depicted. The opening assembly 60 may comprise an opening body 61 which may further comprise LEDs 63-65 with various wavelengths. The opening assembly 60 may further comprise the proximity sensing engine 66. The proximity sensing engine 66 may comprise at least 4 capacitive sensors as shown in the figure.

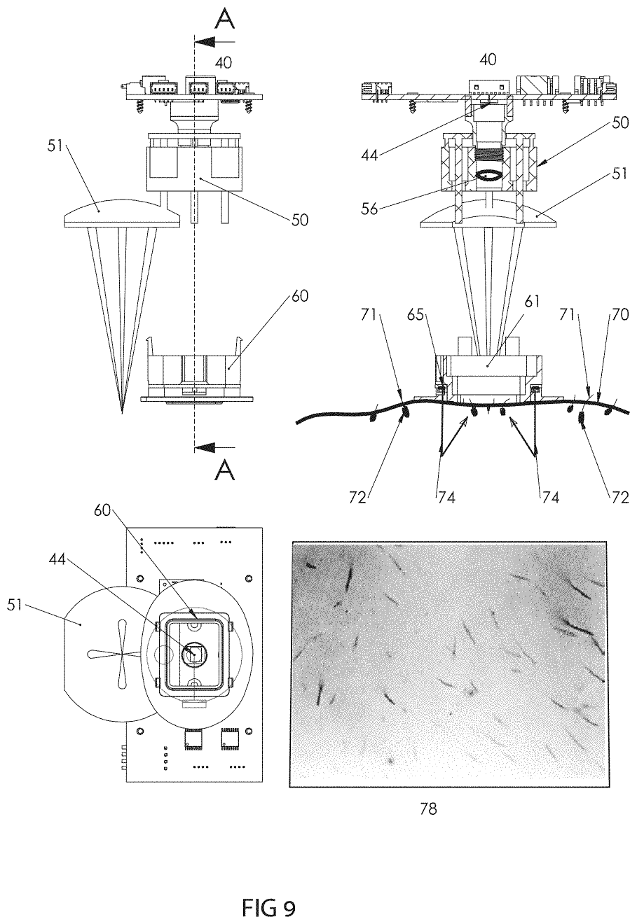

FIG. 9 shows an embodiment of the targeting system. The LED 63-65 transmits light into the skin. The light is reflected from the body 69 and reflected back to the opening 68. The back light shows the shadow 78 of the target area. The processing component 45 receives the image. Each target gets a position in X Y coordinates. Then the X direction motor and the Y direction motor move the focal point to the target position.

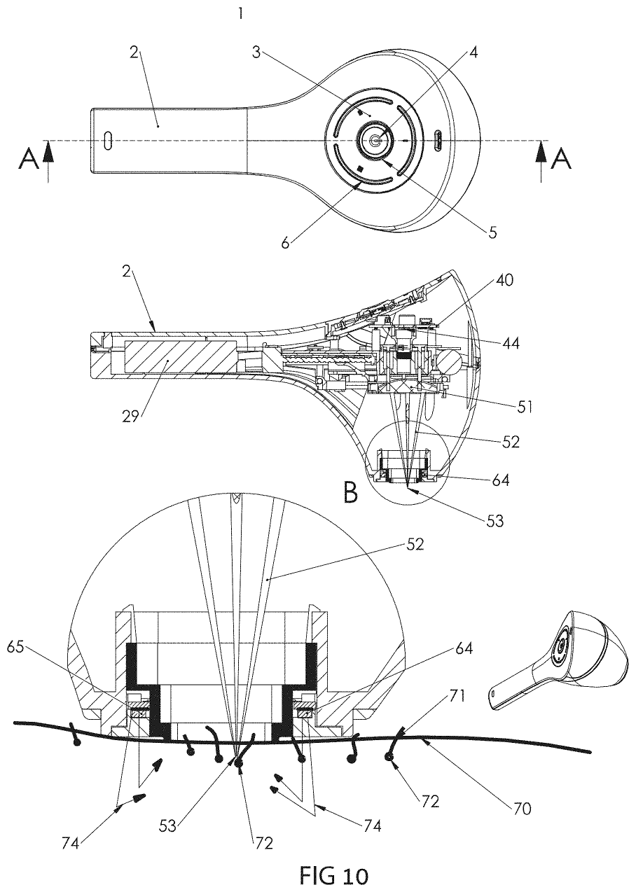

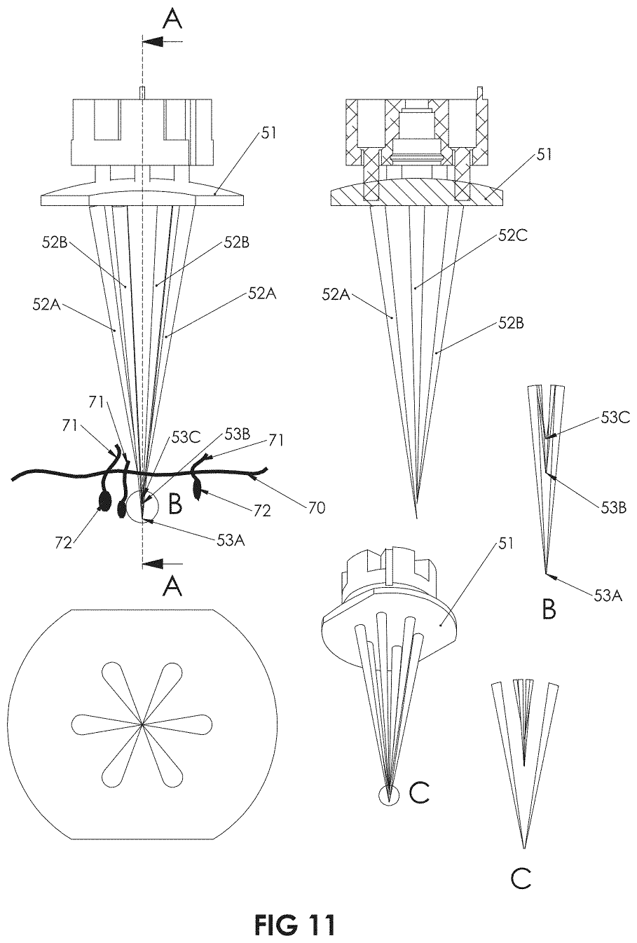

In FIG. 10 and FIG. 11 the device is shown according to an embodiment. The figure shows a deep focus B which may be achieved by multiple lasers 50 working as pairs and disposed on the lens 51 peripheral area. Each pair 53A, 53B, 53C may have two lasers which may be opposite to each other and have a focal point at a certain position along the optical axis A. A relatively long focal area B may be created by combining several wavelengths. The long focal area may increase the efficacy of laser strength.

Reference numbers and letters appearing between parentheses in the claims, identifying features described in the embodiments and illustrated in the accompanying drawings, are provided as an aid to the reader as an exemplification of the matter claimed. The inclusion of such reference numbers and letters is not to be interpreted as placing any limitations on the scope of the claims.

The term “at least one of a first option and a second option” is intended to mean the first option or the second option or the first option and the second option.

Whenever a relative term, such as “about”, “substantially” or “approximately” is used in this specification, such a term should also be construed to also include the exact term. That is, e.g., “substantially straight” should be construed to also include “(exactly) straight”.

Whenever steps were recited in the above or also in the appended claims, it should be noted that the order in which the steps are recited in this text may be accidental. That is, unless otherwise specified or unless clear to the skilled person, the order in which steps are recited may be accidental. That is, when the present document states. e.g., that a method comprises steps (A) and (B), this does not necessarily mean that step (A) precedes step (B), but it is also possible that step (A) is performed (at least partly) simultaneously with step (B) or that step (B) precedes step (A). Furthermore, when a step (X) is said to precede another step (Z), this does not imply that there is no step between steps (X) and (Z). That is, step (X) preceding step (Z) encompasses the situation that step (X) is performed directly before step (Z), but also the situation that (X) is performed before one or more steps (Y1), . . . , followed by step (Z). Corresponding considerations apply when terms like “after” or “before” are used.

1. A system configured to safely localize a target, comprises:
a device comprising a laser system comprising a plurality of laser sources;
an apparatus configured to generate proximity data;
a processing component configured to control the laser system based on target data.

2. The system according to claim 1 wherein the system comprises an adjustment engine configured to move an optical axis of a lens system relative to the laser system, wherein the lens system is configured to focus at least two radiation beams of the laser system.

3. The system according to claim 1 wherein the system further comprises a targeting system, wherein the targeting system is configured to emit at least one or a plurality of EM radiations.

4. The system according to claim 1 wherein the system further comprises an image sensing system, wherein the image sensing system is configured to receive the EM radiations reflected by the target.

5. The system according to claim 4 wherein the image sensing system is configured to generate target data based on the reflected EM radiations.

6. The system according to claim 4 wherein the processing component is configured to pull the target data and adjust at least one of the wavelengths and the fluence of each laser sources based on target data.

7. The system according to claim 4 wherein the system further comprises the processing component pulling the proximity data from a proximity sensing engine, wherein the proximity sensing engine is configured to generate the proximity data based on at least one of at least gravity and at least magnetic orientation and at least distance of an aperture from the target.

8. The system according to claim 7 wherein the proximity sensing engine may be installed at the aperture, wherein the aperture comprises an opening configured to let the laser radiation exit.

9. The system according to claim 7 further comprising the processing component enabling the laser system and the adjustment engine based on at least one of at least the target data and at least the proximity data.

10. The system according to claim 7 wherein each laser sources comprises different wavelength, configured to generate a focal area along the optical axis after passing through the lens.

11. The system according to claim 7 wherein the target data comprises at least one of a position, a type and a colour of the target.

12. A method for localizing a target, comprising:
automatically identifying target data;
generating a plurality of radiations based on the target data;
adjusting the radiations to focus on the target;
enabling the radiations only when a safety value is received.

13. The method according to claim 12 further comprising the step of generating the plurality of radiations via a laser system configured with a plurality of laser sources comprising different wavelengths.

14. The method according to claim 12 further comprising the step of generating the safety value based on a proximity with a target area.

15. The method according to claim 12 wherein the method further comprises the step of focusing the radiations to a plurality of focal points along an optical axis of a lens system.
